(19) United States
(12) Patent Application Publication
(10) Pub. No.: US 2014/0319331 A1
(43) Pub. Date: Oct. 30, 2014

(54) NEW USE FOR A COMPOUND AS A MATRIX IN THE SPECIFIC DETECTION, IDENTIFICATION AND/OR QUANTIFICATION OF ALKALOIDS BY MALDI-TOF MASS SPECTROMETRY

(71) Applicants: Marylène Dias, Soulaire et Bourg (FR); Eric Levillain, Soulaire et Bourg (FR); Pascal Richomme, Angers (FR); Andreas Schinkovitz, Angers (FR); Denis Seraphin, Saint-Saturnin-sur-Loire (FR)

(72) Inventors: Marylène Dias, Soulaire et Bourg (FR); Eric Levillain, Soulaire et Bourg (FR); Pascal Richomme, Angers (FR); Andreas Schinkovitz, Angers (FR); Denis Seraphin, Saint-Saturnin-sur-Loire (FR)

(73) Assignees: CENTRE NATIONAL DE LA RECHERCHE SCIENTIFIQUE CNRS, Paris Cedex 16 (FR); UNIVERSITE D'ANGERS, Angers (FR)

(21) Appl. No.: 14/233,794

(22) PCT Filed: Jul. 16, 2012
(86) PCT No.: PCT/FR2012/051687

§ 371 Date: May 28, 2014

(30) Foreign Application Priority Data
Jul. 18, 2011 (FR) 1156523

(51) Int. Cl. C07D 333/34; H01J 49/00

(52) U.S. Cl. CPC C07D 333/34; H01J 49/0027
USPC 250/282; 549/59

(57) ABSTRACT

There is provided (i) a method of analysing small molecules that may have a mass of <800 Da, in particular alkaloids, said method being generally referred to as MALDI-TOF-MS (or MALDI time-of-flight mass spectrometry), which is an acronym for a method of analysis by matrix-assisted laser desorption/ionisation time-of-flight mass spectrometry. Also provided is (ii) a molecule according to formula (I) and the use of the molecule as a matrix in the analysis method.

### CROSS-REFERENCE TO RELATED APPLICATIONS

This is a U.S. national stage of application No. PCT/FR2012/051687 filed on Jul. 16, 2012. Priority under 35 U.S.C. §119(a) and 35 U.S.C. §365(b) is claimed from French Application No. 1156523, filed Jul. 18, 2011, entire disclosure of which is also incorporated herein by reference.

### TECHNICAL FIELD

The present invention relates (i) to a method for analysis of molecules with a low mass, of possibly <800 Da, alkaloids in particular, said method being generally denoted by the name MALDI-TOF-MS, which is an acronym for method of analysis by matrix-assisted laser desorption/ionization time-of-flight mass spectrometry, and (ii) to a new use of a compound as matrix in such a method of analysis.

In this method, laser pulses are concentrated on a flat sample containing the molecules for analysis, which are incorporated in a matrix. The matrix absorbs the major part of the energy of the laser and subsequently, by the transfer of protons, ionizes and vaporizes the molecules for analysis.

In the description below, the references between square brackets ([ ]) referred to the list of references presented at the end of the text.

### TECHNICAL BACKGROUND

A MALDI-TOF-MS instrument is a mass spectrometer coupling a matrix-assisted laser ionization source, abbreviated MALDI according to the acronym Matrix-Assisted Laser Desorption/Ionization, with a time-of-flight analyzer, abbreviated TOF according to the acronym Time-Of-Flight. It allows the performance of analytical methods that are defined hereinafter.

The MALDI-TOF-MS analytical method is known to find increased use in the determination of the mass of nonvolatile macromolecules.

The reason is that matrix-assisted laser desorption/ionization is a gentle ionization technique which is used in mass spectrometry, permitting the analysis of biomolecules, such as biopolymers, for instance proteins, peptides, and carbohydrates, and of organic molecules such as polymers, dendrimers, and other macromolecules, which have a tendency to become fragile and to break up when ionized by more conventional methods. The MALDI technique has thus allowed the field of application of mass spectrometry to be extended. Traditionally, the MALDI-TOF-MS method is not suitable for the analysis of molecules with low masses, more particularly having a m/z<800.

Ionization is brought about by a laser beam, normally a nitrogen laser. The laser is directed onto the crystals forming the matrix of the MALDI spot which absorbs the laser energy, the matrix being used in order to protect the molecules for analysis from destruction by a direct beam, and to facilitate vaporization and ionization.

The matrix is ionized first of all by this phenomenon, and subsequently transfers part of its charge to the molecules for analysis, causing them to ionize while protecting them from disruptive energy of the laser. The process of bringing the ions into equilibrium takes approximately 100 ns or less, after which the majority of the ions have left the surface with a certain average speed. The result is a scatter in the ion departure time. To compensate this scatter and thereby enhance the mass resolution, extraction of the ions from the source toward the analyzer is delayed for a few hundred nanoseconds, or even a few microseconds. This technique is known by the terminology “delayed extraction” for desorption/ionization.

Time-of-flight mass spectrometry, abbreviated TOF-MS according to the acronym Time of Flight Mass Spectrometry, is a method of analysis in mass spectrometry in which the ions are accelerated by an electrical field of known value. The result of this acceleration is that ions with the same electrical charge acquire the same kinetic energy. The speed of the ions, in contrast, is dependent on the mass/charge ratio. A measurement is made of the time taken for a charged particle to reach a detector situated at a known distance. This time will depend on the mass/charge ratio of the particle in question. The heaviest particles will be accelerated to the lowest speeds. The determination of the mass/charge ratio results from this time of flight and from knowledge of other experimental parameters, such as the position of the detector and the acceleration voltage.

One of the important features of mass spectrometry is the sharpness of the peaks, measured by the resolution of the mass spectrometer. The resolution is defined as being the ratio of the mass m of the peak to the width at half-maximum Δm. The higher the resolution, the sharper the peaks. It is then possible to visualize two molecules with similar masses.

MALDI-TOF-MS instruments may be equipped with a reflectron (electrostatic mirror or “ion mirror”) which turns round the ions with an electrical field, approximately doubling the length of the flight path of the ion, and enhancing the resolution of the instrument. A MALDI-TOF mass spectrometer may attain resolutions of 5000 in linear mode (without reflectron) and of 20 000 with reflectron.

Time-of-flight analyzers, which require a pulsed ionization source, were historically coupled solely with MALDI sources. At present, the MALDI sources may be coupled to other types of analyzers (for example, an FT-ICR analyzer), and the time-of-flight analyzer may be coupled to other sources (for example, with an electrospray source in an ESI-QTOF instrument).

Further explanations associated with the MALDI-TOF-MS method may be found in the following articles: U.S. Pat. No. 6,104,028; The Scientist 13 [12]: 18, Jun. 7, 1999; and Biophotonics International, June 2001, 42-47.

Moreover, the nature of the matrix proves very important in the MALDI-TOF-MS analytical method. The matrix is generally a small organic molecule which is able to absorb the intense laser radiation, thereby preventing decomposition of the molecules for analysis, but which is also able to softly transfer the energy to the molecules for analysis and therefore promote their ionization.

The nature of the molecules for producing an effective matrix is determined by trial-and-error, but remains based on specific considerations of “molecular profiles”. Said molecules:
are required to be of low molecular mass so as to facilitate vaporization, but sufficiently bulky, with a sufficiently high vapor pressure, so as not to evaporate when the sample is being prepared or when it is introduced into the spectrometer;
are required to be acidic, or at least to act as a source of proton(s) so as to promote ionization of the molecules for analysis;
are required to exhibit high absorption in the ultraviolet, allowing them to absorb the laser irradiation efficiently and rapidly; and
are required to be functionalized with polar groups, for use in aqueous solutions.

In a first phase, a matrix in solution in a solvent or in water is mixed with the sample for analysis. An organic solvent allows hydrophobic molecules to dissolve in the solution, while water allows hydrophilic molecules to do likewise. A solution with one of these molecules is produced, sometimes in a mixture of ultrapure water and organic solvent, commonly acetonitrile, abbreviated ACN, or ethanol, abbreviated EtOH. Trifluoroacetic acid, abbreviated TFA, may sometimes be added. A good example of a matrix solution is, for instance, a mixture of 20 mg/ml sinapic acid in an ACN/water/TFA mixture (50/50/0.1 by volume).

Subsequently, in a second phase, the resulting solution is applied to a MALDI cup, commonly made of metal and designed for this use. Said application is carried out according to various application techniques, manual or automated, which also become very significant with regard to the quality of the spectra obtained. The solvent vaporizes at ambient temperature under reduced pressure, leaving the recrystallized matrix, but now with the molecules for analysis distributed throughout the crystal. The matrix and the compound or compounds for analysis are then said to be cocrystallized in a MALDI spot.

A list of examples of compounds which can be used as a matrix, depending on the molecules for analysis, is set out in table 1 below. A given matrix may prove specific to certain molecules it is desired to detect, identify or quantify.

TABLE 1

| Compound | Abbreviation | Use |
|---|---|---|
| 9-Nitroanthracene | 9-NA | Fullerenes |
| Alpha-cyano-4-hydroxycinnamic | HCCA | Peptides of less |
| acid | | than 10 kDa |
| | | Carbohydrates |
| Sinapic acid | SA | Proteins of more |
| | | than 10 kDa |
| | | Fullerenes |
| 2-(4-Hydroxyphenylazo)benzoic | HABA | Proteins of more |
| acid | | than 10 kDa |
| 2,4,6-Trihydroxyacetophenone | THAP | Oligonucleotides of |
| | | less than 3.5 kDa |
| | | Acid carbohydrates |
| 3-Hydroxypicolinic acid | HPA | Oligonucleotides of |
| | | more than 3.5 kDa |
| Anthranilic acid | — | Oligonucleotides of |
| | | more than 3.5 kDa |
| Nicotinic acid | — | Oligonucleotides of |
| | | more than 3.5 kDa |
| trans-3-Indoleacrylic acid | IAA | Nonpolar synthetic |
| | | polymers |
| Dithranol | DIT | Nonpolar synthetic |
| | | polymers |
| | | Lipids |
| 2,5-Dihydroxybenzoic acid | DHB | Polar synthetic polymers |
| | | Organic molecules |
| | | Carbohydrates |
| 1-Isoquinolinol | — | Oligosaccharides |
| Picolinic acid | — | Oligonucleotides |
| 2,5-Dihydroxyacetophenone | DHAP | Proteins of more |
| | | than 10 kDa |

The matrix consists of crystallized molecules which are, in general, selected from the following three compounds: 3,5-dimethoxy-4-hydroxycinnamic acid (sinapic acid/sinapinic acid), alpha-cyano-4-hydroxycinnamic acid (alpha-cyano or alpha-matrix), and 2,5-dihydroxybenzoic acid, abbreviated DHB.

In MALDI-TOF-MS, although the matrix plays an essential part during the desorption/ionization process, it also represents a limiting factor for the technique, in so far as very often, and in substantial quantities, it generates ions in the range of low mass-to-charge (m/z) ratios. This phenomenon therefore greatly complicates the characterization of small molecules, typically <800 Da.

Consequently there is a genuine need for a matrix that overcomes the drawbacks and obstacles of the prior art, and more particularly for a process allowing the analysis of small molecules <800 Da, such as alkaloids, for example, more particularly allowing them to be characterized and/or quantified on the basis of a complex mixture.

Document CN 101644694 A describes the direct detection by MALDI-TOF-MS of alkaloids (aconitine, berberine, strychnine) in crude extracts obtained from various plants of the traditional Chinese Pharmacopeia: Radix aconiti, Rhizoma typhoni, Cortex phellodendri. The DHB matrix used is conventional and not specific to the alkaloids. Moreover, the analysis process disclosed in said document does not allow the quantification of the molecules detected.

The document “Journal of Mass Spectrometry”; 2007; 42: 58-69 describes a method of direct determination of alkaloid profiles in plant tissues such as Chinese herbs, using MALDI-TOF mass spectrometry. Alpha-cyano-4-hydroxycinnamic acid, abbreviated CHCA, and 2,5-dihydroxybenzoic acid, abbreviated DHB, matrices are cited.

The document “Analytica Chimica Acta”; 649 (2009), 230-235 relates to a 7-mercapto-4-methylcoumarin matrix which is appropriate for MALDI analysis of low-molecular-weight compounds, such as carcinogenic alkaloids, more particularly the alkaloids arecoline and arecaidine.

### SUMMARY

The present invention therefore resides in the discovery of new molecules and in their use as a MALDI-TOF matrix, or the use of a molecule which is otherwise known, 3-(5-(5-(methylthio)thiophen-2-yl)thiophen-2-ylthio)propanenitrile, abbreviated MT3P, which is, however, unknown in the prior art for use in MALDI-TOF-MS, these molecules having the advantage of generating very few “parasitic” ions and being able to result in the ionization of small molecules, advantageously of molecular mass—abbreviated MM—<800 Da, more particularly of alkaloid molecules. Since said matrix exhibits a very high sensitivity and/or selectivity, it further enhances the quantification of said small molecules relative to cases where the prior-art matrices are used. With the matrix according to the invention, therefore, it is possible to carry out characterization and quantification of molecules of MM<800 Da that are of very great pharmacological interest, including from complex mixtures such as crude plant extracts, biological fluids, and so on.

The invention accordingly provides the use of the compound of formula (I):

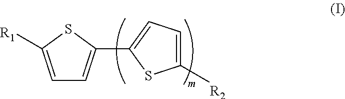

wherein m is 1 or 2, the group R1 is selected from groups —S(CH₂)ₙ—Y where n is an integer selected from 1, 2, 3, and 4 and Y is a functional group selected from —CN, —CO₂R3, and —OH, with R3 being a hydrogen atom or an alkyl group, such as, for example, a C1-C6 alkyl group, and
wherein the group R2 is selected from a hydrogen atom, —Salkyl groups, such as, for example, a C1-C6 alkyl group, such as, for example, the —SC₂H₅ group or the —SCH₃ group, —SCH₂cycloalkyl groups, such as, for example, the —SCH₂C₆H₁₁ group or the —SCH₂C₅H₉ group, and —SCH₂aryl groups, such as, for example, the —SCH₂C₆H₅ group,
as matrix in a matrix-assisted laser desorption/ionization, or MALDI, mass spectrometry device,

The invention likewise provides a compound of formula (I):

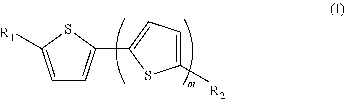

wherein m is 1 or 2, the group R1 is selected from groups —S(CH₂)ₙ—Y where n is an integer selected from 1, 2, 3, and 4 and Y is a functional group selected from —CN, —CO₂R3, and —OH, with R3 being a hydrogen atom or an alkyl group, such as, for example, a C1-C6 alkyl group, and
wherein the group R2 is selected from —Salkyl groups, —SCH₂alkyl groups, such as, for example, a C1-C6 alkyl group, such as, for example, the —SC₂H₅ group or the —SCH₃ group, —SCH₂cycloalkyl groups, such as, for example, the —SCH₂C₆H₁₁ group or the —SCH₂C₅H₉ group, and —SCH₂aryl groups, such as, for example, the —SCH₂C₆H₅ group,
or wherein m=1, R1 is a —S(CH₂)₂CN group, and R2 is a hydrogen atom; with the exception of 3-(5-(5-(methylthio)thiophen-2-yl)thiophen-2-ylthio)-propanenitrile, abbreviated MT3P.

Moreover, the invention also embraces the use of a compound as defined above or of 3-(5-(5-(methylthio)thiophen-2-yl)thiophen-2-ylthio)propanenitrile for producing a matrix intended for use in a matrix-assisted laser desorption/ionization, or MALDI, mass spectrometry device.

Furthermore, the invention relates to a process for producing a matrix intended for use in a matrix-assisted laser desorption/ionization, or MALDI, mass spectrometry device, which comprises crystallizing a compound as defined above or the compound 3-(5-(5-(methylthio)thiophen-2-yl)thiophen-2-ylthio)propanenitrile.

Lastly, the invention likewise relates to a process for characterizing and/or quantifying molecules, advantageously having a mass <800 Da, present in a sample, by matrix-assisted laser desorption/ionization time-of-flight mass spectrometry, comprising a step of:
producing a matrix with a compound as defined above or with a 3-(5-(5-(methylthio)thiophen-2-yl)thiophen-2-ylthio)propanenitrile compound;
mixing said sample with said matrix, optionally in the presence of an organic solvent or water;
vaporizing the solvent or the water, as appropriate;
cocrystallizing said matrix and said molecules, and forming a matrix crystal comprising said molecules distributed throughout said crystal;
subjecting said matrix cocrystallized with the sample, obtained beforehand, to ionization by a laser beam;
establishing a spectrogram;
   it being possible for said sample to consist of epidermal derivatives or extracts thereof, or of a biological fluid selected from blood, plasma, genital mucosae fluids, skin fluids, effusive and closed-cavity fluids, and fluids of the digestive and urinary system.

### BRIEF DESCRIPTION OF THE DRAWINGS

FIG. 1 represents the Mass spectrum (LDI) of MT3P (I), recorded in linear mode (Biflex III, Bruker) for a laser intensity of 10%. The majority ions correspond first to the pseudomolecular ion [M+H]⁺ (m/z 297.018) and secondly to the fragment [(M+H)—C₃H₄N]⁺ (m/z 243.009).

FIG. 2 represents the MALDI-TOF spectrum of a crude extract (EtOH/H₃O⁺) of Senecio vulgaris (Asteraceae) with MT3P (1) as matrix.

FIG. 3 represents, for codeine (2), the chart of I=f(C), where I is the intensity of the pseudomolecular ion and C is the nanomolar concentration of the analyte, plotted on the basis of MALDI-TOF spectra, with MT3P as matrix.

FIG. 4 represents, for hyoscyamine (3), the chart of I=f(C), where I is the intensity of the pseudomolecular ion and C is the nanomolar concentration of the analyte, plotted on the basis of MALDI-TOF spectra, with MT3P as matrix.

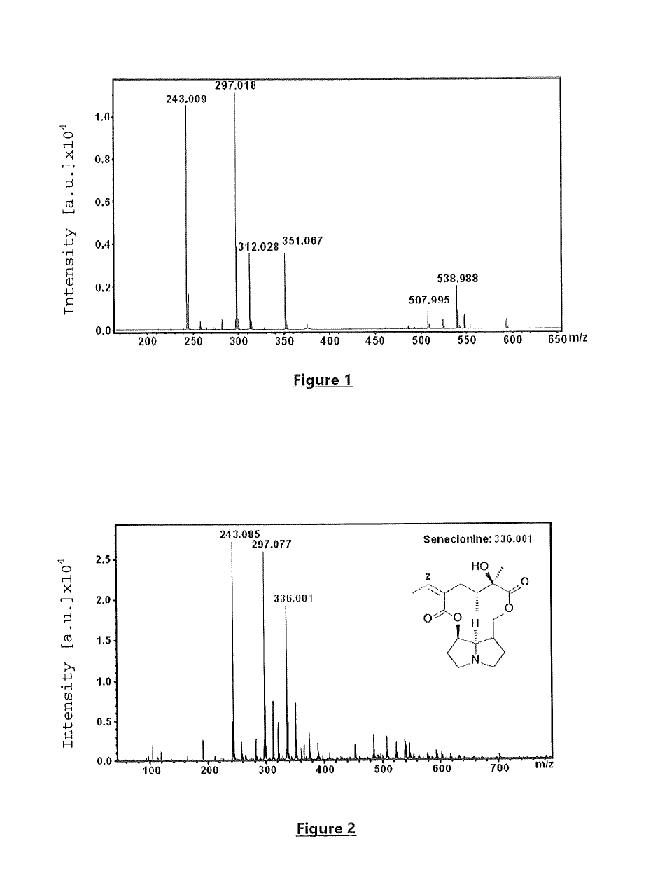

### DETAILED DESCRIPTION OF EMBODIMENTS

In the context of the invention, the meanings of terms are as follows:

“crude organic extract”: any extract obtained from dry or fresh matter originating from living beings such as, for example, Archaea, Bacteria, Protists, Fungi, Plants, and Animals, that has not been subject to any subsequent purification step. Crude plant extracts are distinguished in particular.

“biological fluid”: all fluids obtained from living beings such as, for example, living beings of human or animal origin. Examples of biological fluid include blood, plasma, urine, sperm, and so on.

“organic solvent”: any hydrocarbon compound used alone or in combination to dissolve one or more products.

“alkaloids”: basic, nitrogen-containing, heterocyclic organic molecules possibly having pharmacological activity. Alkaloids are commonly amino acid derivatives. Alkaloids are found, as secondary metabolites, primarily in plants, fungi, and some few animal groups. As well as alkaloids of natural origin, there are also synthetic alkaloids produced by synthesis, such as chloroquine, chloroquinine, or by semisynthesis. There are, for example, alkaloids of the type containing two nitrogen atoms in the aromatic ring and not of natural origin, this being the group of the pyrazoles.

Although many alkaloids are toxic (such as strychnine or aconitine), some are employed in medicine by virtue, for example, of their analgesic properties (such as morphine, codeine), as part of sedation protocols (anesthesia), often accompanied by hypnotics, or as antimalarial agents (quinine, chloroquinine) or anticancer agents (vinblastine, vincristine).

“alkyl group”: a linear or branched aliphatic hydrocarbon group comprising, for example, one carbon atom (abbreviated C1) to six carbon atoms (abbreviated C6).

“cycloalkyl group”: a cyclic alkyl, in other words an aliphatic and cyclic hydrocarbon group comprising, for example, from three carbon atoms (abbreviated C3) to six carbon atoms (abbreviated C6).

“aryl group”: an aromatic group which may comprise at least one heteroatom.

The invention accordingly provides the use of the compound of formula (I):

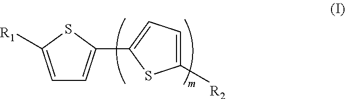

wherein m is 1 or 2, the group R1 is selected from groups —S(CH₂)ₙ—Y where n is an integer selected from 1, 2, 3, and 4 and Y is a functional group selected from —CN, —CO₂R3, and —OH, with R3 being a hydrogen atom or a C1-C6 alkyl group, and
wherein the group R2 is selected from a hydrogen atom, —Salkyl groups, —SCH₂cycloalkyl groups, and —SCH₂aryl groups, such as, for example, —SCH₃, —SC₂H₅, and —SCH₂C₆H₅ groups,
as matrix in a matrix-assisted laser desorption/ionization, or MALDI, mass spectrometry device. The MALDI device may be coupled to a time-of-flight analyzer.

According to one embodiment, R2 is the —SCH₃ group and/or n is 2 and/or Y is a —CN functional group and/or m is 1.

Advantageously, the compounds of formula (I) in question are those wherein:
m=1, R1 is a —S(CH₂)₂CN group, and R2 is a —SCH₃ group;
m=1, R1 is a —S(CH₂)₂CO₂CH₃ group, and R2 is a —SCH₃ group;
m=1, R1 is a —S(CH₂)₃CN group, and R2 is a —SCH₃ group;
m=1, R1 is a —S(CH₂)₂CN group, and R2 is a hydrogen atom;
m=1, R1 is a —S(CH₂)₂CO₂H group, and R2 is a —SCH₃ group;
m=1, R1 is a —S(CH₂)₂—CH₂OH group, and R2 is a —SCH₃ group;
m=1, R1 is a —S(CH₂)₂N₃ group, and R2 is a —SCH₃ group; or
m=1, R1 is a —S(CH₂)₂CCH group, and R2 is a —SCH₃ group.

According to one particularly preferred embodiment, the compound of formula (I) is 3-(5-(5-(methylthio)thiophen-2-yl)thiophen-2-ylthio)propanenitrile or MT3P.

Such a use is particularly advantageous for quantitative and qualitative analysis of a molecule of mass <800 Da or a mixture of molecules or a sample of crude organic extract or of biological fluid, said mixture or said sample comprising at least one molecule of mass <800 Da, it being possible for said molecule of mass <800 Da to be selected from alkaloids as defined hereinafter.

The alkaloids are categorized according to their chemical structure. The following are distinguished:

Group of the Azolidines (pyrrolidines): Aniracetam, Anisomycin, CX614, Dextromoramide, Diphenylprolinol, domoic acid, Histapyrrodine, kainic acid, Methdilazine, Oxaceprol, Prolintane, Pyrrobutamine, hygrine, cuscohygrine.

Group of the Azines: Piperidine, Conicine, Trigonelline, Arecaidine, Guvacine, Pilocarpine, Cytisine, Nicotine, Sparteine, Pelletierine.

Group of the Tropanes: Atropine, Hyoscyamine, Cocaine, Ecgonine, Scopolamine.

Group of the Quinolines: Acridine, Bicinchoninic acid, Broxyquinoline, Chlorquinaldol, Cinchophen, Clioquinol, Dequalinium, Dihydroquinine, Dihydroquinidine, Hydroxychloroquine, 8-Hydroxyquinoline, Iodoquinol, Kynurenic acid, Mefloquine, Nitroxoline, Oxycinchophen, Primaquine, Quinine, Quinidine, TSQ, Topotecan, xanthurenic acid, Strychnine, Brucine, Veratrine, Cevadine, Echinopsine;
Aminoquinolines: Chloroquine, Hydroxychloroquine, Primaquine;
8-Aminoquinolines: Tafenoquine, Rhodoquine, Pamaquine.

Group of the Isoquinolines: Dimethisoquine, Quinapril, Quinapirilat, Debrisoquine, 2,2′-Hexadecamethylenediisoquinolinium dichloride, N-laurylisoquinolinium bromide, Narceine, Hydrastine, Berberine;
Opium alkaloids:

Natural: Morphine, Codeine, Thebaine, Papaverine, Narcotine, Noscapine;

Semisynthetic: Hydromorphone, Hydrocodone, Heroin;

Synthetic: Fentanyl, Pethidine, Methadone, Propoxyphene.

Group of the indoles:
Ergolines: Rye ergot alkaloids (Ergometrine, Ergotamine, Ergosine, Ergovaline, Ergokryptine, Ergocornine, Ergocristine, lysergic acid, etc.), Ergine, LSD, and so on;
Beta-carbolines: Harmine, Yohimbine, Reserpine, Emetine.

Group of the Terpenoids:
Aconite alkaloids: Aconitine;
Solanidine, Solasodine, Batrachotoxin, Delphinine;
Steroids: Solanine, Samandarin.

Group of the Betaines (quaternary ammonium compounds, not alkaloids in the true sense, although regularly classed as such): Muscarine, Choline, Neurine.

Group of the Pyrazoles.

Said molecule of mass <800 Da may be selected from 12-Demethylthalrugosidine, Aconitine, Atropine, Berberine, Boldine, Cholchicine, Clavuline, Codeine, Emetine, Fumaritine, Harmine, L-Hyoscyamine, Limogine, Morphine, Nicotine, Pilocarpine, Quinidine, Senecionine, Sparteine, Strychnine, Stylopine, Thalfoetidine, Thaliberine, Thaliglucinone, and Yohimbine.

The molecule or molecules of mass <800 Da and/or the alkaloid molecule or molecules defined above may thus be detected and quantified in samples of biological fluids or of crude organic extracts, more particularly of crude plant extracts, without requiring prior steps of purification or separation, by matrix-assisted laser mass spectrometry.

According to one embodiment, the sample is selected from crude organic extracts, or any fraction obtained from said crude organic extracts, and biological fluids or samples, of human or animal origin. The biological samples include, for example, epidermal derivatives such as the nails or the hair.

The biological fluid may be selected from blood, plasma, liquids of the genital mucosae, seminal fluid, sperm, cervical mucus, skin fluids such as perspiration, effusive and closed-cavity fluids, and fluids of the digestive and urinary system.

The invention likewise provides a compound of formula (I):

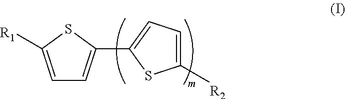

wherein m is 1 or 2, the group R1 is selected from groups —S(CH₂)ₙ—Y where n is an integer selected from 1, 2, 3, and 4 and Y is a functional group selected from —CN, —CO₂R3, and —OH, with R3 being a hydrogen atom or a C1-C6 alkyl group, and
wherein the group R2 is selected from —Salkyl groups, —SCH₂alkyl groups, —SCH₂cycloalkyl groups, and —SCH₂aryl groups, such as, for example, the —SCH₃ group, the —SC₂H₅ group, and the —SCH₂C₆H₅ group,
or wherein m=1, R1 is a —S(CH₂)₂CN group, and R2 is a hydrogen atom; with the exception of 3-(5-(5-(methylthio)thiophen-2-yl)thiophen-2-ylthio)-propanenitrile.

Advantageously, R2 is the —SCH₃ group and/or n is 2 and/or Y is a —CN functional group and/or m is 1 in said compound.

According to one embodiment, the compounds in question are those in which:

m=1, R1 is a —S(CH₂)₂CO₂CH₃ group, and R2 is a —SCH₃ group;

m=1, R1 is a —S(CH₂)₃CN group, and R2 is a —SCH₃ group;

m=1, R1 is a —S(CH₂)₂CO₂H group, and R2 is a —SCH₃ group;

m=1, R1 is a —S(CH₂)₂—CH₂OH group, and R2 is a —SCH₃ group;

m=1, R1 is a —S(CH₂)₂N₃ group, and R2 is a —SCH₃ group; or

m=1, R1 is a —S(CH₂)₂CCH group, and R2 is a —SCH₃ group.

The invention also relates to a process for producing a matrix intended for use in a matrix-assisted laser desorption/ionization, or MALDI, mass spectrometry device, which comprises crystallizing a compound as defined above or the compound 3-(5-(5-(methylthio)thiophen-2-yl)thiophen-2-ylthio)propanenitrile. Said MALDI device may be a mass spectrometer coupling a matrix-assisted laser ionization source and a time-of-flight analyzer.

According to one embodiment, said process is characterized in that said compound as defined above, in crystallized form, or said 3-(5-(5-(methylthio)thiophen-2-yl)thiophen-2-ylthio)propanenitrile compound that has been crystallized, is subsequently optionally dissolved in an organic solvent or water comprising a sample for analysis, or dissolved directly in the sample for analysis, said solvent or water being subsequently, where appropriate, vaporized, ultimately forming a crystallized matrix as defined above or of 3-(5-(5-(methylthio)thiophen-2-yl)thiophen-2-ylthio)propanenitrile compound comprising the sample for analysis.

The sample may be selected from crude organic extracts and biological fluids.

This process may take place with a molar (molecules for analysis)/(molecules of matrix) ratio of between 1/30 and 1/50, preferably 1/39.

MT3P (1) has the following characteristics, which it endows on the matrix that it forms or in the composition of which it forms part:
MT3P (1) requires a low laser irradiation intensity (5% to 10%) for the desorption/ionization process, causing little fragmentation of the analyte and of the matrix, and producing MALDI-TOF mass spectra which are characterized by high signal-to-noise (S/N) ratios;
MT3P (1) exhibits a very high selectivity, especially with regard to alkaloids, which are nitrogen-containing heterocycles generally featuring marked pharmacological activities. Accordingly, the use of MT3P (1) does not lead to any probative result in the case of derivatives such as steroids (pregnolone, digitoxin), coumarins (imperatorin, E-notopterol), polyphenols (rutin, amentoflavone), carotenoids (tocotrienols), peptides (angiotensin II or glycerides (1,3-dimethylglycerol, glycerol 1,3-distearate)).

On the contrary, alkaloids, irrespective of their basic structure (indoles, (iso)quinolines, quinolizidines, pyrrolizidines, tropanes, etc.) are detected readily in the very great majority of cases, under the same conditions, as is illustrated in table 2 below. The resulting spectra then present, for the quasimolecular ions, S/N ratios which are equivalent to or greater than those obtained in the case where conventional matrices are used, such as 2-cyano-3-(4-hydroxyphenyl)propanoic acid (HCCA), ditranol, or 2,5-dihydroxybenzoic acid (DHB). This excellent detection sensitivity allied with a high specificity for analytes of interest thus permits, for example, the direct characterization, from a crude organic extract with acidic ethanol of Senecio vulgaris, of senecionine, a derivative of retronicine that is mutagenic, teratogenic, and induces hepatic tumors, as is shown in the spectrum in FIG. 2. The presence of senecionine has to be looked for in a variety of food ingredients such as the oil from viper's bugloss, Echium plantagineum [regulation (EC) No. 258/97 of the European Parliament and of the Council of Jan. 27, 1997; Scientific Document: Opinion of the Panel on contaminants in the food chain [CONTAM] related to pyrrolizidine alkaloids as undesirable substances in animal feed, The EFSA Journal, 447, 1-51 (2007)].

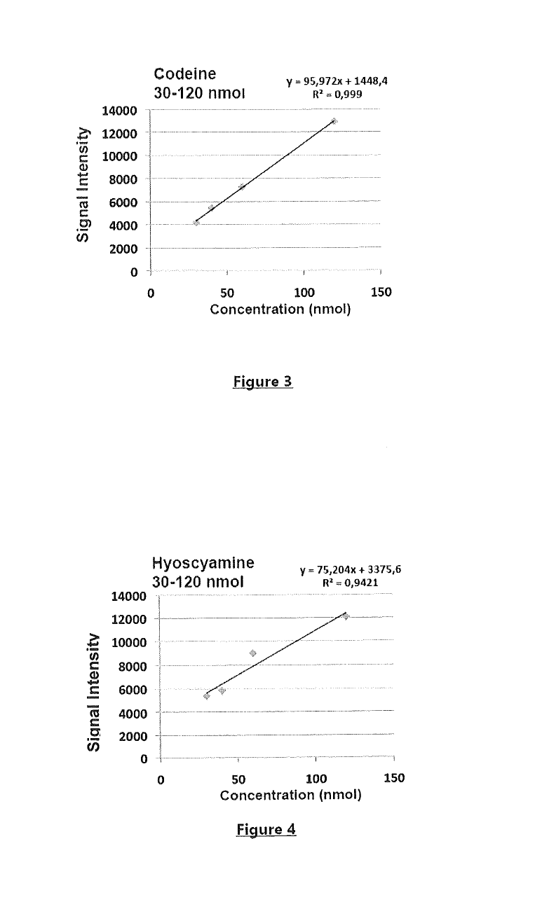

Consequently, a very large number of (semi)quantitative analyses can be carried out with a matrix of or comprising MT3P (1). Depending on the quality of the charts obtained, indeed, it is possible to develop alkaloid-specific assay methods that exploit the very high sensitivity of the MALDI-TOF mass spectrometers, leading to limits of detection (LOD) or of quantification (LOQ) that are very low, as shown in FIGS. 3 and 4. This is the case for codeine (2), an analgesic and antitussive opioid, or else for hyoscyamine (3), a mydriatic alkaloid, which are shown in scheme 1 below.

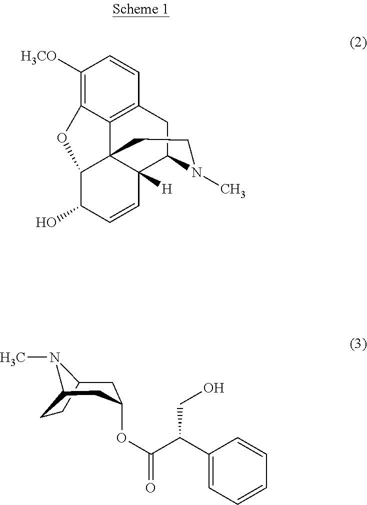

The 3-(5-(5-(methylthio)thiophen-2-yl)thiophen-2-ylthio)propanenitrile matrix according to the invention has the advantage, moreover, that it can be prepared in three simple steps, with a good yield of the order of 80% by weight, from commercial products, and therefore at moderate cost.

Furthermore, it exhibits a unique selectivity for alkaloids, thereby permitting their characterization and their (semi)quantification by MALDI-TOF-MS from complex mixtures, without special preparation of the samples, and with excellent parameters for limit of detection or LOD and for limit of quantification or LOQ.

MT3P consists of a chromophore organized around a planar conjugated system, and absorbs at the wavelength (337 nm) of the N₂ lasers most commonly used in MALDI-TOF MS, on the one hand, and is provided, on the other hand, with a nitrile function, via a spacer, this function being capable of developing dipolar interactions with the amine or imine functions of the compounds sought. It should be noted, lastly, that the use of MT3P requires only low laser irradiation powers, and promotes the protonation of the analytes.

It should also be emphasized that the protocols for alkaloid assay or characterization that are drawn up in this way do not necessitate any modification at all to the MALDI-TOF spectrometers with which the majority of analytical platforms are nowadays equipped.

In the context of the new REACH regulations, therefore, the use of this matrix may represent a valuable diagnostic tool for studies of pharmacovigilance or toxicology.

Other advantages, such as the exploitation of MT3P in MALDI imaging, may further become apparent to the skilled person from a reading of the examples below, which are illustrated by the attached figures and which are given for illustration.

### EXAMPLES

### Example 1

### Preparation of the MT3P (1) Matrix

The compound 3-(5-(5-(methylthio)thiophen-2-yl)thiophen-2-ylthio)-propanenitrile (1) or MT3P (1) below is prepared from 2-bromothiophene in three steps and with an overall yield of 80%, as shown according to scheme 2 below. The reaction conditions of this synthesis are as follows:
for step i) magnesium, NidpppCl₂, Et₂O, reflux;
for step ii) nBuLi, sulfur, and 2-bromopropionitrile, THF, room temperature;
for step iii) cesium hydroxide and iodomethane, DMF/MeOH, room temperature.
   The detail of these steps of this synthesis is described in the document Mass Spectrom., 2006; 41: 830-833.

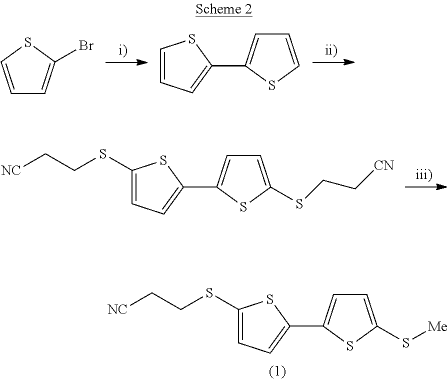 [SMILES: Brc1cccs1 ; c1csc(-c2cccs2)c1 ; N#CCCSc1ccc(-c2ccc(SCCC#N)s2)s1 ; CSc1ccc(-c2ccc(SCCC#N)s2)s1]

### Example 2

### Preparation of the Sample for Analysis

The analyte is dissolved in a suitable organic solvent (e.g. CH₂Cl₂, MeOH, etc.) at a concentration of 2.57 mmol/L. The solution prepared is stored at a temperature of −20° C. Prior to each experiment carried out in MALDI/TOF (Preparation of the matrix cocrystallized with the sample, as explained in the paragraph which follows), this solution is brought to room temperature and then diluted 1:3 in MeOH.

### Example 3

### Preparation of the Matrix Cocrystallized with the Sample for Analysis

The cocrystallized matrix intended for use in a matrix-assisted laser desorption/ionization, or MALDI, mass spectrometry device, for a single analyte, is prepared as follows: One equivalent of analyte solution at a concentration of 0.861 mmol is mixed with two equivalents of matrix solution at a concentration of 33.67 mmol. The final concentrations are 0.287 mmol/L for the analyte and 11.22 mmol/L for the matrix. For a crude extract, for example, it is possible to mix one equivalent of crude extract (22 mg/mL) with two equivalents of concentrated matrix (30 mg/mL=0.1 mmol/mL).

### Example 4

### Alkaloid Characterization Tests by MALDI-TOF MS with MT3P (1) as Matrix

### Method of Calibration of the Spectrometer:

The spectrometer is calibrated with 20 microliters of a mixture of PEG400/MeOH in proportions by volume of 1/3. This solution is mixed with 20 microliters of Ditranol at a concentration of 1010 mg/mL.

0.7 microliters of this solution are applied to a MALDI cup. The sample is irradiated with a laser at an energy level between 35-40%. The PEG-Na signals observed are used for the automated calibration. Agreement is observed between the signals observed and the data for PEG-Na mass in high resolution.

The alkaloids tested are assembled in table 2 below.

### Analytical Parameters of the Spectrometer:

### the Experimental Parameters of MALDI-TOF-MS:

Ion source 1: 19.00 kV,

Ion source 2: 17.35 kV,

Lenses: 9.60 kV,

Ion extraction pulse: 200 ns,

Laser frequency: 5 ns,

Voltage offset of detector gain: 1300 V,

Electronic gain: 100 mV,

Acquisition window: 20-2000 Da

TABLE 2

| | | Monoisotopic | |
|---|---|---|---|
| Alkaloid | Formula | mass | Detection |
| 12- | C₃₇H₄₀N₂O₇ | 624.28 | + |
| Demethylthalrugosidine | | | |
| Aconitine | C₃₄H₄₇NO₁₁ | 645.31 | + + |
| Atropine | C₁₇H₂₃NO₃ | 289.17 | + + |
| Berberine | C₂₀H₁₈NO₄ | 336.12 | + + + |
| Boldine | C₁₉H₂₁NO₄ | 327.14 | + + |
| Cholchicine | C₂₂H₂₅NO₆ | 399.17 | + + |
| Clavuline | C₁₈H₁₉NO₄ | 313.13 | + + + |
| Codeine | C₁₈H₂₁NO₃ | 299.15 | + + |
| Emetine | C₂₉H₄₀N₂O₄ | 480.30 | + + + |
| Fumaritine | C₂₀H₂₁NO₅ | 355.14 | + + + |
| Harmine | C₁₃H₁₂N₂O | 212.09 | + + + |
| L-Hyoscyamine | C₁₇H₂₃NO₃ | 289.17 | + + |
| Limogine | C₂₀H₁₇NO₅ | 351.11 | + + + |
| Morphine | C₁₇H₁₉NO₃ | 285.14 | + + |
| Nicotine | C₁₀H₁₄N₂ | 162.12 | − |
| Pilocarpine | C₁₁H₁₆N₂O₂ | 208.13 | + + |
| Quinidine | C₂₀H₂₄N₂O₂ | 324.18 | + + + |
| Senecionine | C₁₈H₂₅NO₅ | 335.17 | + + |
| Sparteine | C₁₅H₂₆N₂ | 234.21 | + + + |
| Strychnine | C₂₁H₂₂N₂O₂ | 334.16 | + + |
| Stylopine | C₁₉H₁₇NO₄ | 323.12 | + + + |
| Thalfoetidine | C₃₈H₄₂N₂O₇ | 638.30 | + |
| Thaliberine | C₃₇H₄₀N₂O₆ | 608.29 | + + + |
| Thaliglucinone | C₂₁H₁₉NO₅ | 365.13 | − |
| Yohimbine | C₂₁H₂₆N₂O₃ | 354.19 | + + |

### Comparatives and Examples 5 to 16

### Characterization Tests on Compounds by MALDI-TOF MS with Different Matrices Conforming or not Conforming to the Invention

For each comparative or example, the sample for analysis and the matrix cocrystallized with the sample for analysis are prepared in the same way as that set out in example 2 and example 3 above.

Moreover, the method of calibration of the spectrometer and the analytical parameters of the spectrometer are identical to those in example 4 above.

The same alkaloids tested on MT3P matrix in example 4 are tested on matrices other than MT3P, conforming or not conforming to the invention.

The detection results of the comparatives, not conforming to the invention, abbreviated Cp6, Cp7, Cp14, Cp15, and Cp16, and the detection results of the examples, conforming to the invention, abbreviated Ex4, Ex5, Ex8, Ex9, Ex10, Ex11, Ex12, and Ex13, are compiled in table 3A below.

Moreover, molecules other than alkaloids are also tested on some of these non-MT3P matrices. The detection results of comparatives 6 and 7 (abbreviated Cp6 to Cp7) and of examples 5 and 8 to 13 (abbreviated Ex5 and Ex8 to Ex13) are compiled in table 3B below.

The signal/noise ratios of the peaks corresponding to the quasimolecular ions are classed as: high: +++; medium: ++; low: +; very low: −; and zero: 0 (nt signifies “not tested”).

TABLE 3A

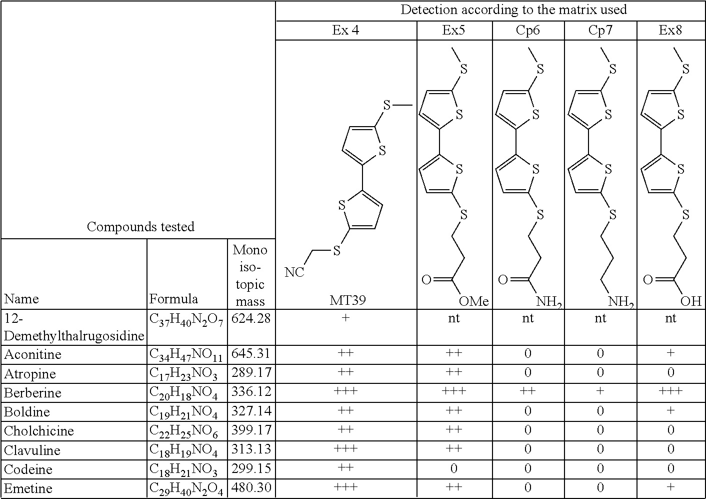

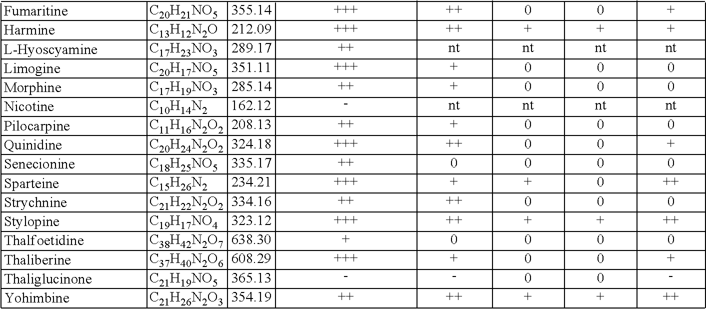

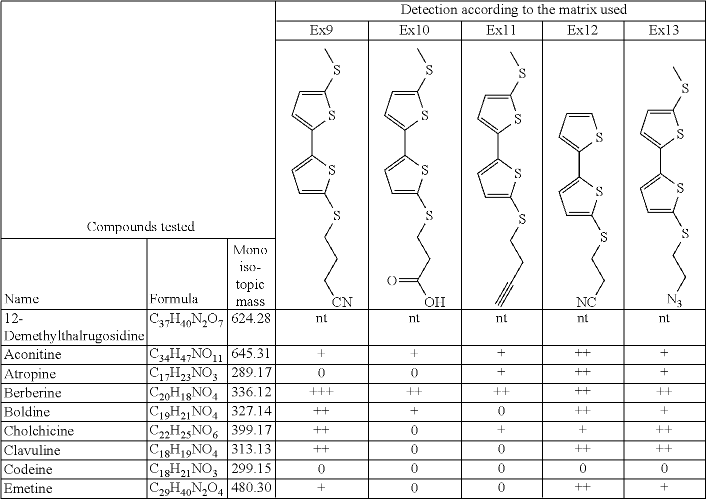

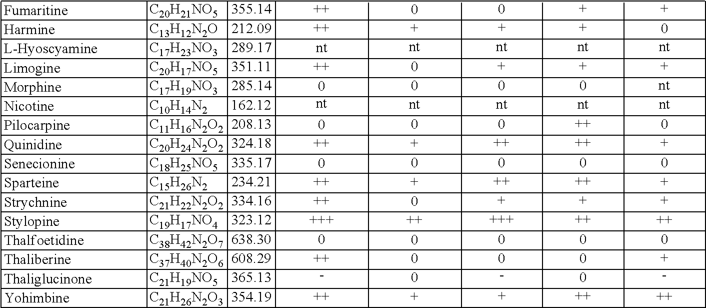

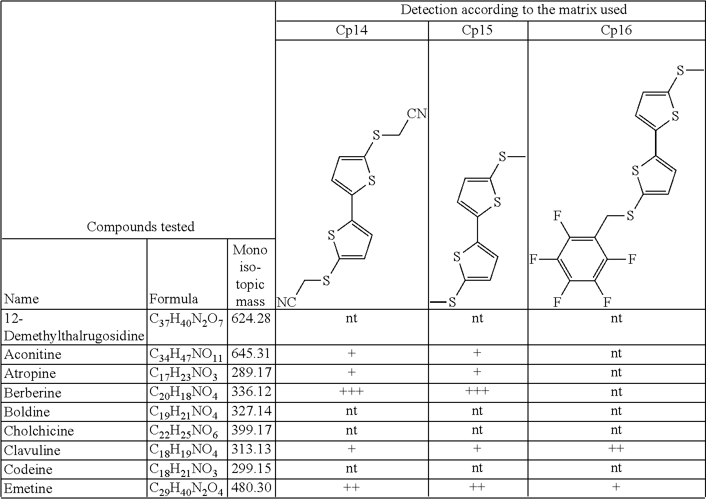

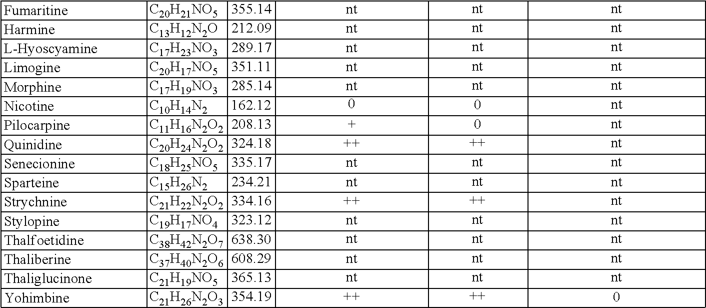

TABLE 3B

| | Detection according to the matrix used | | | | | | | | |
|---|---|---|---|---|---|---|---|---|---|
| Compounds tested | Ex5 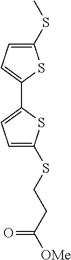 [SMILES: COC(=O)CCSc1ccc(-c2ccc(SC)s2)s1] | Cp6 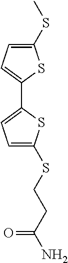 [SMILES: CSc1ccc(-c2ccc(SCCC(N)=O)s2)s1] | Cp7 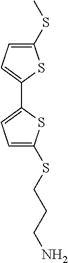 [SMILES: CSc1ccc(-c2ccc(SCCCN)s2)s1] | Ex8 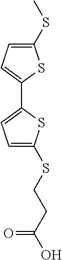 [SMILES: CSc1ccc(-c2ccc(SCCC(=O)O)s2)s1] | Ex9 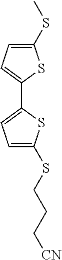 [SMILES: CSc1ccc(-c2ccc(SCCCC#N)s2)s1] | Ex10 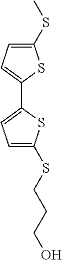 [SMILES: CSc1ccc(-c2ccc(SCCCO)s2)s1] | Ex11 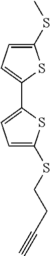 [SMILES: C#CCCSc1ccc(-c2ccc(SC)s2)s1] | Ex12 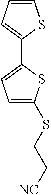 [SMILES: N#CCCSc1ccc(-c2cccs2)s1] | Ex13 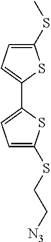 [SMILES: CSc1ccc(-c2ccc(SCCN=[N+]=[N-])s2)s1] |
| 1,3-Dipalmitoylglycerol | 0 | 0 | 0 | 0 | 0 | 0 | 0 | 0 | 0 |
| Acetylsalicylic acid | 0 | 0 | 0 | 0 | 0 | 0 | 0 | 0 | 0 |
| Caffeic acid | — | 0 | — | 0 | 0 | 0 | 0 | 0 | 0 |
| Chlorogenic acid | 0 | 0 | 0 | 0 | 0 | 0 | 0 | 0 | 0 |
| Fumaric acid | 0 | — | — | 0 | 0 | 0 | 0 | 0 | 0 |
| Amentoflavone | 0 | 0 | 0 | 0 | 0 | 0 | nt | 0 | 0 |
| Angiotensin II | 0 | 0 | 0 | 0 | 0 | 0 | 0 | 0 | 0 |
| Bergaptene | 0 | 0 | 0 | 0 | 0 | 0 | 0 | 0 | 0 |
| Caryophyllene | 0 | 0 | 0 | 0 | 0 | 0 | 0 | 0 | 0 |
| Coumarin | 0 | 0 | 0 | 0 | 0 | 0 | 0 | 0 | 0 |
| Curcumin | 0 | 0 | 0 | 0 | 0 | 0 | 0 | 0 | nt |
| Digitoxin | 0 | 0 | 0 | 0 | 0 | 0 | 0 | 0 | 0 |
| E-Notopterol | 0 | 0 | 0 | 0 | 0 | 0 | 0 | 0 | 0 |
| Hesperidine | 0 | 0 | 0 | 0 | 0 | 0 | 0 | 0 | 0 |
| Isoimperatorine | 0 | 0 | 0 | 0 | 0 | 0 | 0 | 0 | 0 |
| Khelline | 0 | 0 | 0 | 0 | 0 | 0 | 0 | 0 | 0 |
| Pentamethoxyflavone | — | 0 | 0 | 0 | 0 | 0 | — | — | — |
| Pregnolone | nt | nt | 0 | nt | 0 | 0 | 0 | 0 | 0 |
| Quercetin | 0 | 0 | 0 | 0 | 0 | 0 | 0 | 0 | 0 |
| Rutin | 0 | 0 | 0 | 0 | 0 | 0 | 0 | nt | 0 |
| Sitosterol | 0 | 0 | 0 | 0 | 0 | 0 | 0 | 0 | 0 |

### LISTS OF REFERENCES

U.S. Pat. No. 6,104,028;
   “The Scientist” 13 [12]; 18, Jun. 7, 1999;
   “Biophotonics International”, June 2001, 42-47.
CN 101644694;
“Journal of mass spectrometry”, 2007; 42; 58-69;
“Analytica Chimica Acta”, 649 (2009), 230-235.

1. The use of the compound of formula (I): 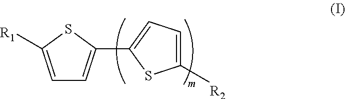
wherein m is 1 or 2, the group R1 is selected from groups —S(CH₂)ₙ—Y where n is an integer selected from 1, 2, 3, and 4 and Y is a functional group selected from —CN, —CO₂R3, and —OH, with R3 being a hydrogen atom or an alkyl group, and
wherein the group R2 is selected from a hydrogen atom, —Salkyl groups, —SCH₂cycloalkyl groups, and —SCH₂aryl groups, as matrix in a matrix-assisted laser desorption/ionization, or MALDI, mass spectrometry device.

2. The use as claimed in claim 1, wherein R2 is the —SCH₃ group and/or n is 2 and/or Y is a —CN functional group and/or m is 1.

3. The use as claimed in claim 1, wherein:
m=1, R1 is a —S(CH₂)₂CN group, and R2 is a —SCH₃ group;
m=1, R1 is a —S(CH₂)₂CO₂CH₃ group, and R2 is a —SCH₃ group;
m=1, R1 is a —S(CH₂)₃CN group, and R2 is a —SCH₃ group;
m=1, R1 is a —S(CH₂)₂CN group, and R2 is hydrogen atom;
m=1, R1 is a —S(CH₂)₂CO₂H group, and R2 is a —SCH₃ group;
m=1, R1 is a —S(CH₂)₂—CH₂OH group, and R2 is a —SCH₃ group;
m=1, R1 is a —S(CH₂)₂N₃ group, and R2 is a —SCH₃ group; or
m=1, R1 is a —S(CH₂)₂CCH group, and R2 is a —SCH₃ group.

4. The use as claimed in claim 1, wherein the compound of formula (I) is 3-(5-(5-(methylthio)thiophen-2-yl)thiophen-2-ylthio)propanenitrile.

5. The use as claimed in claim 1, wherein said MALDI device is coupled to a time-of-flight analyzer.

6. The use as claimed in claim 1, for qualitative and quantitative analysis of a mixture of molecules or sample comprising at least one molecule of mass <800 Da.

7. The use as claimed in claim 1, wherein said molecule of mass <800 Da is selected from alkaloids.

8. The use as claimed in claim 7, wherein the alkaloids are selected from:
the group of the Azolidines;
the group of the Azines;
the group of the Tropanes;
the group of the Quinolines;
the group of the Isoquinolines;
the group of the Indoles;
the group of the Terpenoids;
the group of the Betaines;
the group of the Pyrazoles.

9. The use as claimed in claim 7, wherein the alkaloids are selected from (i) Aniracetam, Anisomycin, CX614, Dextromoramide, Diphenylprolinol, Domoic acid, Histapyrrodine, Kainic acid, Methdilazine, Oxaceprol, Prolintane, Pyrrobutamine, Hygrine, Cuscohygrine; (ii) Piperidine, Conicine, Trigonelline, Arecaidine, Guvacine, Pilocarpine, Cytisine, Nicotine, Sparteine, Pelletierine; (iii) atropine, L-Hyoscyamine, Cocaine, Ecgonine, Scopolamine; (iv) Acridine, Bicinchoninic acid, Broxyquinoline, Chlorquinaldol, Cinchophen, Clioquinol, Dequalinium, Dihydroquinine, Dihydroquinidine, Hydroxychloroquine, 8-Hydroxyquinoline, Iodoquinol, Kynurenic acid, Mefloquine, Nitroxoline, Oxycinchophen, Primaquine, Quinine, Quinidine, TSQ, Topotecan, Xanthurenic acid, Strychnine, Brucine, Veratrine, Cevadine, Echinopsine, Aminoquinolines such as Chloroquine, Hydroxychloroquine, Primaquine, 8-Aminoquinolines such as Tafenoquine, Rhodoquine, Pamaquine; (v) Dimethisoquine, Quinapril, Quinapirilat, Debrisoquine, 2,2′-Hexadecamethylenediisoquinolinium dichloride, N-Laurylisoquinolinium bromide, Narceine, Hydrastine, Berberine, natural opium alkaloids such as Morphine, Codeine, Thebaine, Papaverine, Narcotine, Noscapine, semisynthetic opium alkaloids such as Hydromorphone, Hydrocodone, Heroin, synthetic opium alkaloids such as Fentanyl, Pethidine, Methadone, Propoxyphene; (vi) Ergolines such as rye ergot alkaloids such as Ergometrine, Ergotamine, Ergosine, Ergovaline, Ergokryptine, Ergocornine, Ergocristine, Lysergic acid, Ergine, LSD, (vii) Beta-carbolines such as Harmine, Yohimbine, Reserpine, Emetine; (viii) aconite alkaloids such as Aconitine, Solanidine, (ix) Solasodine, Batrachotoxin, Delphinine, (x) steroids such as Solanine, Samandarin; (xi) Muscarine, Choline, Neurine.

10. The use as claimed in claim 7, wherein said molecule of mass <800 Da is selected from 12-Demethylthalrugosidine, Aconitine, Atropine, Berberine, Boldine, Cholchicine, Clavuline, Codeine, Emetine, Fumaritine, Harmine, L-Hyoscyamine, Limogine, Morphine, Nicotine, Pilocarpine, Quinidine, Senecionine, Sparteine, Strychnine, Stylopine, Thalfoetidine, Thaliberine, Thaliglucinone, and Yohimbine.

11. The use as claimed in claim 6, wherein said mixture or sample is a mixture selected from crude organic extracts, or any fraction obtained from said crude organic extracts, and biological fluids, of human or animal origin.

12. The use as claimed in claim 11, wherein said sample is selected from epidermal derivatives, blood and plasma, fluids of the genital mucosae, fluids of the skin, effusive fluids and closed-cavity fluids, and fluids of the digestive and urinary system.

13. A compound of formula (I): 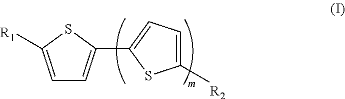
wherein m is 1 or 2, the group R1 is selected from groups —S(CH₂)ₙ—Y where n is an integer selected from 1, 2, 3, and 4 and Y is a functional group selected from —CN, —CO₂R3, and —OH, with R3 being a hydrogen atom or an alkyl group, and
wherein the group R2 is selected from —Salkyl groups, —SCH₂alkyl groups, —SCH₂cycloalkyl groups, and —SCH₂aryl groups,
or wherein m=1, R1 is a —S(CH₂)₂CN group, and R2 is a hydrogen atom;
with the exception of 3-(5-(5-(methylthio)thiophen-2-yl)thiophen-2-ylthio)propanenitrile.

14. The compound as claimed in claim 13, in which R2 is the —SCH₃ group and/or n is 2 and/or Y is a —CN functional group and/or m is 1.

15. The compound as claimed in claim 13, in which:
m=1, R1 is a —S(CH₂)₂CO₂CH₃ group, and R2 is a —SCH₃ group;
m=1, R1 is a —S(CH₂)₃CN group, and R2 is a —SCH₃ group;
m=1, R1 is a —S(CH₂)₂CO₂H group, and R2 is a —SCH₃ group;
m=1, R1 is a —S(CH₂)₂—CH₂OH group, and R2 is a —SCH₃ group;
m=1, R1 is a —S(CH₂)₂N₃ group, and R2 is a —SCH₃ group; or
m=1, R1 is a —S(CH₂)₂CCH group, and R2 is a —SCH₃ group.

16. The use of a compound as claimed in claim 13 or 3-(5-(5-(methylthio)thiophen-2-yl)thiophen-2-ylthio)propanenitrile for producing a matrix intended for use in a matrix-assisted laser desorption/ionization, or MALDI, mass spectrometry device.

17. A process for producing a matrix intended for use in a matrix-assisted laser desorption/ionization, or MALDI, mass spectrometry device, which comprises crystallizing a compound as claimed in claim 13 or the compound 3-(5-(5-(methylthio)thiophen-2-yl)thiophen-2-ylthio)propanenitrile.

18. The process as claimed in claim 17, wherein said MALDI device is a mass spectrometer coupling a matrix-assisted laser ionization source and a time-of-flight analyzer.

19. The process as claimed in claim 17, characterized in that said compound is subsequently dissolved in an organic solvent or water comprising a sample for analysis, said solvent or water being subsequently vaporized to form eventually a crystallized matrix of said compound comprising the sample for analysis.

20. The process as claimed in claim 19, wherein the sample is selected from crude organic extracts, or any fraction obtained from crude organic extracts, and biological fluids, of human or animal origin.

21. The process as claimed in claim 19, wherein the molar (molecules for analysis)/(molecules of matrix) ratio is between 1/30 and 1/50, preferably 1/39.

22. A process for characterizing and/or quantifying molecules, advantageously having a mass <800 Da, present in a sample, by matrix-assisted laser desorption/ionization time-of-flight mass spectrometry, comprising a step of:
producing a matrix with a compound as claimed in claim 13 or with a 3-(5-(5-(methylthio)thiophen-2-yl)thiophen-2-ylthio)propanenitrile compound;
mixing said sample with said matrix, optionally in the presence of an organic solvent or water;
vaporizing the solvent or the water, as appropriate;
cocrystallizing said matrix and said molecules, and forming a matrix crystal comprising said molecules distributed throughout said crystal;
subjecting said matrix cocrystallized with the sample, obtained beforehand, to ionization by a laser beam;
establishing a spectrogram.

23. The process as claimed in claim 22, characterized in that the sample is selected from crude organic extracts, or any fraction obtained from said crude organic extracts, and biological fluids or samples, of human or animal origin.

24. The process as claimed in claim 22, characterized in that the sample is selected from blood, plasma, fluids of the genital mucosae, fluids of the skin, effusive and closed-cavity fluids, fluids of the digestive and urinary system, and epidermal derivatives.
